(19) United States
(12) Patent Application Publication
(10) Pub. No.: US 2026/0182937 A1
(43) Pub. Date: Jul. 2, 2026

(54) METHOD AND APPARATUS FOR PERFORMING PRECISE PATIENT-SPECIFIC AUTOMATIC TUBE CURRENT MODULATION IN COMPUTED TOMOGRAPHY

(71) Applicant: CANON MEDICAL SYSTEMS CORPORATION, Tochigi (JP)

(72) Inventors: Yujie LU, Vernon Hills, IL (US); Ting XIA, Vernon Hills, IL (US); Jian ZHOU, Vernon Hills, IL (US); Liang CAI, Vernon Hills, IL (US)

(73) Assignee: CANON MEDICAL SYSTEMS CORPORATION, Tochigi (JP)

(21) Appl. No.: 19/004,003

(22) Filed: Dec. 27, 2024

(51) Int. Cl. A61B 6/00; A61B 6/42

(52) U.S. Cl. CPC A61B 6/542; A61B 6/4241; A61B 6/488; A61B 6/5205; A61B 6/5223; A61B 6/5258

(57) ABSTRACT

An apparatus for performing X-ray exposure control in a computed tomography (CT) imaging system including an X-ray source is provided. The apparatus includes processing circuitry configured to acquire scout scan data from a scout scan performed on an imaging object, in a slice-by-slice manner, generate a plurality of view-group-based noise images for slices of the CT imaging system, based on the acquired scout scan data, identify an ROI with respect to which the X-ray exposure control is to be performed, determine a target noise SD to be achieved by performing the X-ray exposure control, generate a tube current modulation curve with respect to the identified ROI, based on the generated plurality of view-group-based noise images and the determined target noise SD, and perform an imaging scan on the imaging object, with a tube current applied to the X-ray source being modulated based on the generated tube current modulation curve.

### BACKGROUND

### Field

This disclosure relates to X-ray computed tomography (CT) imaging.

### Description of the Related Art

Computed tomography (CT) scans use ionizing radiation to produce images of patients' bodies, which can increase the risk of developing cancer over time. Studies have shown that CT scans contribute the highest collective amount of medical radiation exposure in the United States compared with any other medical imaging modality.

When conducting diagnostic CT imaging, depending on different protocols, different anatomies of the body may require different exposure levels. It is ideal to scan patients with minimized dose while keeping image quality at a clinically acceptable level. However, reducing the dose often leads to a low signal-to-noise ratio (SNR), potentially affecting the detectability of certain structures or pathologies.

One effective solution to address this problem is automatic exposure control (AEC), a method of adjusting the X-ray tube current in real-time. AEC aims to automatically optimize CT scan exposures, reducing the radiation dose to the body while ensuring a consistent image quality, so as to simplify radiologists' workflow. This strategy has been widely adopted across various protocols and anatomical regions in routine clinical practice.

Almost all CT vendors now offer AEC functionality in clinical scans. Recent advancements include organ-based tube current modulation to mitigate radiation exposure to sensitive organs. However, current AEC approaches usually rely on 2D radiographic images (typically one or two projection views), which provide limited predictive accuracy due to insufficient tomographic image information. Variations in patient size, anatomy, and location within the CT scanner during scanning further increase the difficulty of AEC prediction.

Additionally, most of the image quality requirements are task-dependent and become more complicated when different protocols are used. Using the limited 2D radiographic images, AEC methods based on simple models or look-up tables cannot achieve the precision required for task-dependent patient-specific AEC.

Therefore, precise and individualized AEC is still a challenging problem, especially for patient-specific scans.

### SUMMARY

The present disclosure relates to an apparatus for performing X-ray exposure control in a computed tomography (CT) imaging system including an X-ray source. The apparatus includes processing circuitry configured to acquire scout scan data from a scout scan performed on an imaging object, in a slice-by-slice manner, generate a plurality of view-group-based noise images for slices of the CT imaging system, based on the acquired scout scan data, identify a region-of-interest (ROI) with respect to which the X-ray exposure control is to be performed, determine a target noise standard deviation (SD) to be achieved by performing the X-ray exposure control, generate a tube current modulation curve with respect to the identified ROI, based on the generated plurality of view-group-based noise images and the determined target noise SD, and perform an imaging scan on the imaging object, with a tube current applied to the X-ray source being modulated based on the generated tube current modulation curve.

The disclosure additionally relates to a method for performing X-ray exposure control in a CT imaging system including an X-ray source. The method includes: acquiring scout scan data from a scout scan performed on an imaging object; in a slice-by-slice manner, generating a plurality of view-group-based noise images for slices of the CT imaging system, based on the acquired scout scan data; identifying an ROI with respect to which the X-ray exposure control is to be performed; determining a target noise SD to be achieved by performing the X-ray exposure control; generating a tube current modulation curve with respect to the identified ROI, based on the generated plurality of view-group-based noise images and the determined target noise SD; and performing an imaging scan on the imaging object, with a tube current applied to the X-ray source being modulated based on the generated tube current modulation curve.

The disclosure additionally relates to a non-transitory computer readable medium having instructions stored therein that, when executed by one or more processors, cause the one or more processors to perform the above-described method for performing X-ray exposure control in a CT imaging system including an X-ray source.

Note that this summary section does not specify every embodiment and/or incrementally novel aspect of the present disclosure or claimed invention. Instead, the summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIG. 1 shows a block diagram of an exemplary apparatus for generating tube current modulation curves in a computed tomography (CT) imaging system in accordance with embodiments of the disclosure;

FIG. 2 shows a flow chart of an exemplary procedure of generating tube current modulation curves in a CT imaging system in accordance with embodiments of the disclosure;

FIG. 3 shows a block diagram of view-group-based noise image generation circuitry in accordance with embodiments of the disclosure;

FIG. 4 shows exemplary noise projections corresponding to multiple view groups, in accordance with embodiments of the disclosure;

FIG. 5 shows a flow chart of an exemplary procedure of generating a plurality of noise images for slices of the CT imaging system, in accordance with embodiments of the disclosure;

FIG. 6 shows a block diagram of region-of-interest (ROI) identification circuitry in accordance with embodiments of the disclosure;

FIG. 7 shows an exemplary ROI identified in a patient image reconstructed from the scout scan data, in accordance with embodiments of the disclosure;

FIG. 8 shows a flow chart of an exemplary procedure of identifying an ROI in accordance with embodiments of the disclosure;

FIG. 9 shows a block diagram of tube current curve generation circuitry in accordance with embodiments of the disclosure;

FIG. 10 shows a flow chart of an exemplary procedure of generating a tube current curve in accordance with embodiments of the disclosure; and

FIG. 11 shows a schematic block diagram of an exemplary CT imaging system that can incorporate the techniques disclosed herein.

### DETAILED DESCRIPTION

The following disclosure provides embodiments or examples for implementing different features of the provided subject matter. Specific examples of components and arrangements are described below to simplify the present disclosure. These are, of course, merely examples and are not intended to be limiting.

For example, the order of discussion of the different steps as described herein has been presented for the sake of clarity. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

Furthermore, as used herein, the words “a,” “an,” and the like generally carry a meaning of “one or more,” unless stated otherwise.

The present disclosure provides a method and apparatus aimed at improving automatic explore control (AEC) accuracy and thereby enhancing the image quality of computed tomography (CT) imaging systems. In a helical CT scan, continuous imaging is achieved by moving the patient through the scanner while the X-ray source and the detector rotate simultaneously, resulting in a spiral-shaped dataset without gaps. Each image slice is reconstructed from hundreds, or even more than a thousand, projection views. Typical AEC methods usually use a single tube current scaling factor shared across all projection views contributing to the same image slice, as they rely on a model that determines an optimal scaling factor per slice. In contrast, the disclosed method and apparatus can determine distinct scaling factors for multiple groups of projection views contributing to each slice. By calculating optimal scaling factors for individual view groups, this AEC approach enables more precise exposure control and enhanced prediction accuracy.

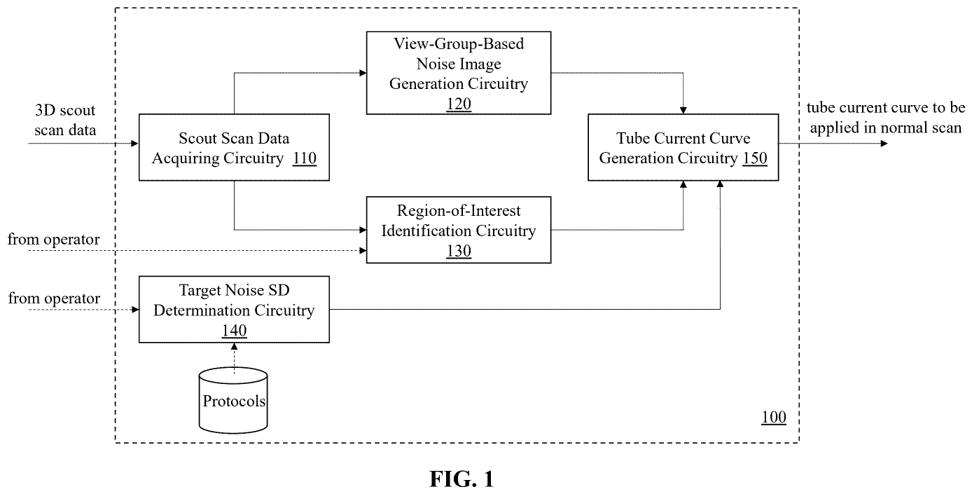

FIG. 1 shows a block diagram of an exemplary apparatus 100 for generating tube current curves in a CT imaging system in accordance with embodiments of the disclosure. The apparatus 100 includes scout scan data acquiring circuitry 110, view-group-based noise image generation circuitry 120, region-of-interest identification (ROI) circuitry 130, target noise standard deviation (SD) determination circuitry 140, and tube current curve generation circuitry 150.

Before conducting a standard (or normal) scan on the patient, a low-dose scout scan can be performed. The scout scan data acquiring circuitry 110 acquires scan data obtained from the scout scan, and sends the acquired data to the view-group-based noise image generation circuitry 120 and the region-of-interest identification circuitry 130. To improve the accuracy of the AEC modulation, a 3D scout scan can be performed to obtain helical scan data, which can provide more comprehensive pre-scan information of the patient.

The view-group-based noise image generation circuitry 120 processes the scout scan data received from the scout scan data acquisition circuitry 110. Based on the scout scan data, the view-group-based noise image generation circuitry 120 generates a plurality of noise images for the individual slices of the CT scanner. These noise images are then sent to the tube current curve generation circuitry 150.

After receiving the scout scan data from the scout scan data acquiring circuitry 110, the ROI identification circuitry 130 identifies a specific ROI with respect to which the AEC is to be performed. The ROI can be positioned in various parts of the patient's body, such as the head, thorax, abdomen, pelvis, etc. Once identified, the ROI is sent to the tube current curve generation circuit 150.

The target noise SD determination circuitry 140 establishes a target noise SD for the normal scan to be performed on the patient after the scout scan, and sends the target noise SD to the tube current curve generation circuitry 150. For instance, the target noise SD can be automatically determined based on the protocol of the normal scan. Alternatively, the target noise SD can be manually set by the operator of the CT scanner. Additionally, the target noise SD can be determined based on the scan protocol, and then refined according to the operator's inputs.

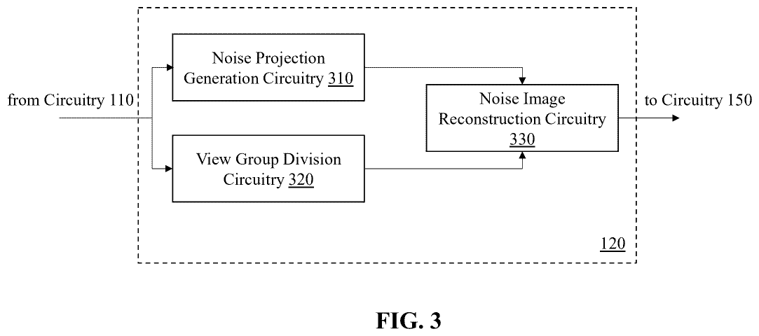

Using the noise images provided by the view-group-based noise image generation circuitry 120, the tube current curve generation circuitry 150 generates a tube current modulation curve to be applied in the normal scan, ensuring that the noise within the identified ROI matches the target noise SD. The detailed structures and functions of the view-group-based noise image generation circuitry 120, ROI identification circuitry 130, and tube current curve generation circuitry 150 will be further described with reference to FIGS. 3-10.

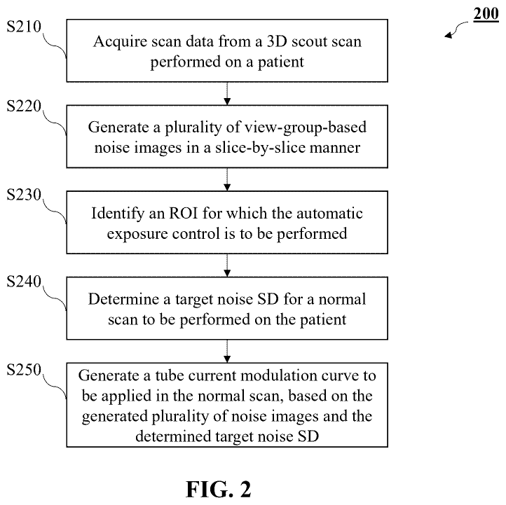

FIG. 2 shows a flow chart of an exemplary procedure of generating tube current modulation curves in a CT imaging system in accordance with embodiments of the disclosure. In step S210, scout scan data from a 3D scout scan is acquired. In step S220, a plurality of view-group-based noise images are generated for the slices of the CT scanners, in a slice-by-slice manner. In step S230, an ROI, where the AEC will be applied, is identified. In step S240, a target noise SD for the normal scan is determined. Finally, in step S250, a tube current modulation curve is generated for the normal scan, based on the noise images and the target noise SD.

FIG. 3 shows a block diagram of the view-group-based noise image generation circuitry 120 in accordance with embodiments of the disclosure. The view-group-based noise image generation circuitry 120 includes noise projection generation circuitry 310, view group division circuitry 320, and noise image reconstruction circuitry 330.

The noise projection generation circuitry 310 receives the scout scan data, and calculates noise projections based on the scout scan data, using a noise model. The calculated noise projections are then sent to the noise image reconstruction circuitry 330.

For example, noise in the measured X-ray photon counts can be modeled using a combination of Poisson and Gaussian distributions. In this case, the variance of the measured count (c) can be expressed as:

$$Var ( c ) = {v}_{1} + {v}_{2} = {a}^{2} λ + {σ}_{e}^{2} \qquad ( 1 )$$

where v₁ and v₂ represent the variances of the photon and electronic noise, respectively, α is the gain of the CT's data acquisition system, λ is the mean of the measured counts for each detector pixel, and

$${σ}_{e}^{2}$$

denotes the variance of the electronic noise. Note that Equation (1) is only one example of the noise model; any other models describing the noise distribution in the scan data also can be used.

The view group division circuitry 320 receives the scout scan data, and divides the views that contribute to each slice into a predetermined number of view groups. The number of view groups can be empirically determined, balancing factors such as computation time, image quality, noise intensity, etc. Although each slice of the CT scanner can be divided into the same number of view groups, different slices can have varying numbers of groups. For instance, if a slice needs finer tube current modulation, more view groups can be used. In contrast, if it is decided that a relatively coarse modulation is sufficient, fewer view groups can be applied to the slice.

In one example, the views that contribute to a single slice can be divided evenly among the predetermined number of groups. For instance, if 700 projection views contributing to a slice are to be divided into 10 groups, 70 views can be assigned to each view group. The views can be assigned sequentially, with the first group including views 1-70, the second group including views 71-140, for example.

Based on the division pattern provided by the view group division circuitry 320, the noise image reconstruction circuitry 330 reconstructs the noise images. For example, analytical reconstruction can be performed on the noise projections. For each slice, noise projections corresponding to the projection views in the first view group are reconstructed into a first noise image; noise projections corresponding to the projection views in the second view group are reconstructed into a second noise image, and so forth.

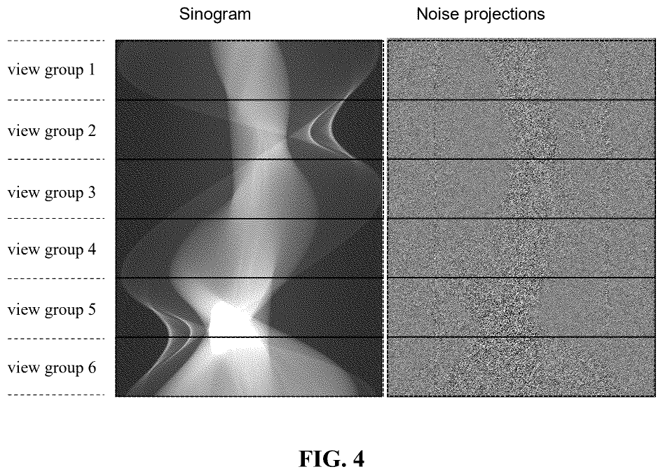

FIG. 4 shows exemplary noise projections generated for a single slice in accordance with embodiments of the disclosure. The left half of the figure shows a sinogram used to reconstruct one image slice, where the entire set of views contributing to this slice is divided into six view groups. Accordingly, the noise projections calculated using Equation (1) are also divided into six corresponding groups, as illustrated in the right half of the figure. As discussed above, each of the six noise projection groups can be reconstructed into a noise image. The combination of these six noise images produces a complete noise image of the slice.

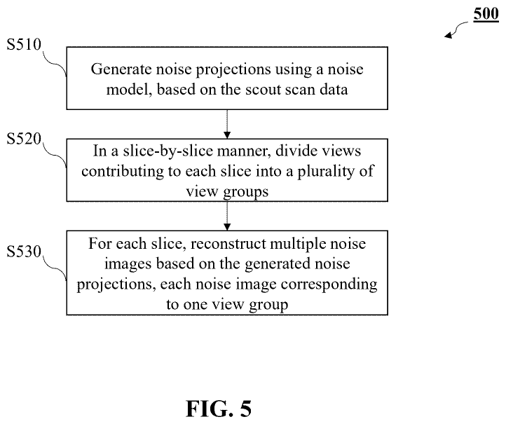

FIG. 5 shows a flow chart of an exemplary procedure of generating a plurality of noise images for the slices of the CT scanner, in accordance with embodiments of the disclosure. In step S510, noise projections are calculated using a noise model, based on the scout scan data. In step S520, views contributing to each slice are divided into a predetermined number of view groups, in a slice-by-slice manner. In step S530, for each slice, multiple partial noise images are reconstructed from the calculated noise projections, with each noise image corresponding to one view group.

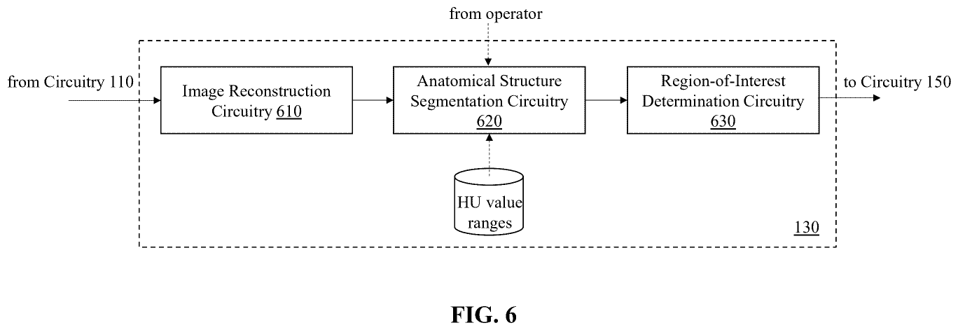

FIG. 6 shows a block diagram of the ROI identification circuitry 130 in accordance with embodiments of the disclosure. The ROI identification circuitry 130 includes image reconstruction circuitry 610, anatomical structure segmentation circuitry 620, and ROI determination circuitry 630.

The image reconstruction circuitry 610 receives the scout scan data from the scout scan data acquiring circuitry 110, and reconstructs the data to generate an image of the patient. Fast reconstruction methods, such as analytical reconstruction, can be used to expedite the process. The resulting patient image contains anatomical information of the patient.

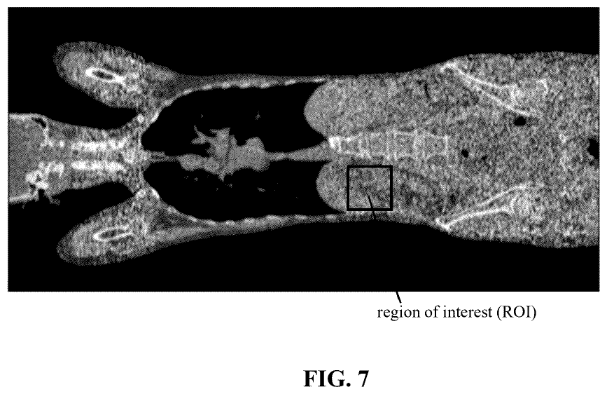

From the reconstructed image, the anatomical structure segmentation circuitry 620 can identify and segment a specific anatomical structure of the patient. Based on inputs from a radiologist, the ROI determination circuitry 630 can select a region around the segmented anatomical structure as the ROI, for example. Additionally, or alternatively, the ROI determination circuitry 630 can automatically define the ROI based on a Hounsfield Unit (HU) value range determined by the scan protocol. An example of an ROI identified from the reconstructed patient image is illustrated in FIG. 7.

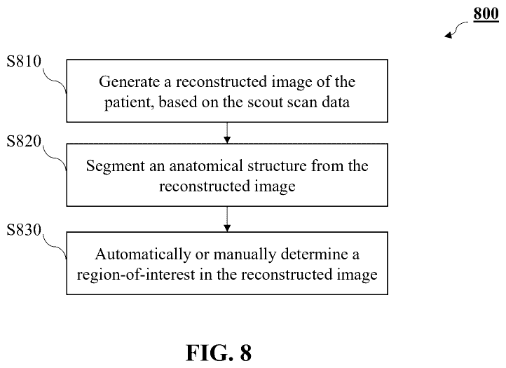

FIG. 8 shows a flow chart of an exemplary procedure of identifying an ROI in accordance with embodiments of the disclosure. In step S810, an image of the patient is generated by performing reconstruction on the scout scan data. In step S820, an anatomical structure is segmented from the reconstructed image. In step S830, an ROI is determined manually based on the operator's selection, or automatically using a HU value range.

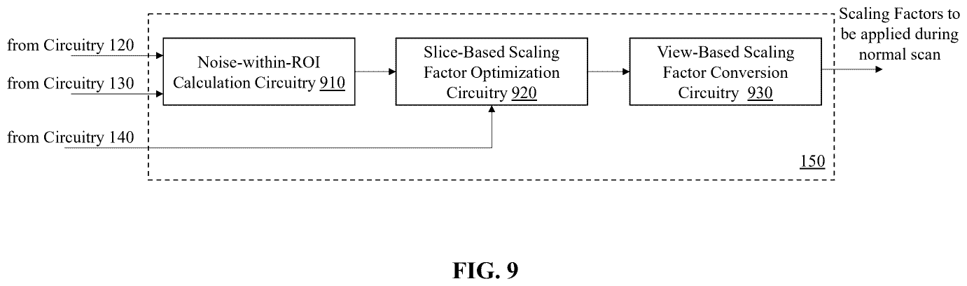

FIG. 9 shows a block diagram of the tube current curve generation circuitry 150 in accordance with embodiments of the disclosure. The tube current curve generation circuitry 150 includes noise-within-ROI calculation circuitry 910, slice-based scaling factor optimization circuitry 920, and view-based scaling factor conversion circuitry 930.

The noise-within-ROI calculation circuitry 910 receives the noise images from the view-group-based noise image generation circuitry 120, and the ROI from the ROI identification circuitry 130. As previously discussed, for each specific slice, multiple partial noise images are reconstructed based on noise projections corresponding to various view groups. These partial noise images combine to provide a complete noise image for the slice.

For instance, in the scenario shown in FIG. 7, six partial noise images can be reconstructed for a slice, with each noise image corresponding to one of the six view groups. For the determined ROI (denoted with Ω), variances v_{i,Ω} in the image domain can be calculated for these partial noise images, where i represents the noise image index, i=1, 2, . . . , 6. The relationship between the noise SD (denoted with s_{Ω}) in the ROI and the calculated variances v_{i,Ω} is given by:

$${s}_{Ω} = \sqrt{{\sum }_{i} {v}_{i , Ω}} \qquad ( 2 )$$

Note that the use of six view groups is merely an example. The number of the view groups can be adjusted empirically, in accordance with the performance of AEC modulation.

Considering two sorts of variances, v₁ and v₂, in the photon and electronical noises, the relationship between s_{Ω}, α, v₁, and v₂ can be established as follows:

$${s}_{Ω} = \sqrt{{\sum }_{i} ( \frac{1}{{α}_{i}} {v}_{i , Ω , 1} + \frac{1}{{α}_{i}^{2}} {v}_{i , Ω , 2} )} \qquad ( 3 )$$

Using the target SD (denoted with s*) provided by the target noise SD determination circuitry 140, a set of optimal scaling factors αᵢ can be derived by solving the following optimization problem:

$$\{ \} = arg {min}_{\{ {α}_{i} > 0 \}} {\sum }_{j} {( {s}^{*} - \sqrt{{\sum }_{i} ( \frac{1}{{α}_{i}} {v}_{i , Ω , 1} + \frac{1}{{α}_{i}^{2}} {v}_{i , Ω , 2} )} )}^{2} \qquad ( 4 )$$

where j represents the image slice index. Let Nₛ be the total number of the image slices, with j=1, 2, . . . , Nₛ.

For example, in the scenario shown in FIG. 7, a set of six optimal scaling factors can be obtained for the slice, with [symbol] corresponding to the first view group, [symbol] corresponding to the second view group, and so forth.

The objective function above is designed to solve an optimal scaling factor for each view group contributing to a slice, such that the difference between the target SD and the noise present in the ROI is minimized. Other suitable objective functions also can be used to achieve this optimization.

Once sets of optimal scaling factors are solved for all individual image slices of the CT scanner, a slice-based scaling factor vector, α, is generated. Each element of this vector corresponds to a specific view group of a specific image slice, with the total number of the elements being the sum of the view groups numbers across all slices of the CT scanner. For example, if there are 700 slices in the CT scanner and each slice has 6 view groups, the resulting scaling factor vector α will have 700×6=4200 elements.

As the tube current modulation is implemented in a view-by-view manner, it is necessary to perform further conversion. A view-based scaling factor vector β is introduced, where each element of the vector β corresponds to a specific view of the CT scanner. The relationship between the slice-based scaling factor vector α and the view-based scaling factor vector β can be defined as follows:

$$β = diag ( \frac{1}{r} ) R α \qquad ( 5 )$$

In Equation (5), R (dimensioned Nᵥ×(Nᵢ×Nₛ)) is a matrix for converting the slice-based vector α into the view-based vector β for implementing the AEC modulation, Nᵥ is the total number of the projection views, Nₛ is the total number of the image slices, Nᵢ is the number of the view groups for each slice. The normalized factor diag

$$( \frac{1}{r} )$$

can be calculated as rᵢ=Σj R(i, j), where i=1, 2, . . . Nᵥ, and j=1, 2, . . . , Nᵢ×Nₛ.

As discussed previously, the entire set of views contributing to a slice is divided into multiple view groups. As each one of the multiple view groups can be reconstructed to an image sub-slice, the matrix R describes the relationship between the total sub-image slices and the projection views. For example, R can be a simple binary matrix. For a specific image sub-slice corresponding to the view group, if a view contributes to that image sub-slice, the element value of R is set at 1, otherwise, the element value is set at 0. Note that other continuous or smoothing weighting factors can be used to form the matrix R. For example, alternative weighting factors, such as continuous or smoothing weights based on view redundancy or weights manually designed based on the shape of the imaging object, can also be used.

Returning to Equation (4), the optimal scaling factors ai are solved through iterative optimization. Since the number of views is usually much larger than the number of ROIs, the solution to the optimization problem may not be unique. To improve stability, the optimization variables can be parametrized using a suitable basis function.

For example, assuming that the scaling factor α changes smoothly, it can be represented as a smooth parametric curve, such as a b-spline function:

$${α}_{i} = {\sum }_{k} {γ}_{k} B ( {α}_{i} - {c}_{k} ) , k = 1 , … , K , ∀ i \qquad ( 6 )$$

Here, B is the b-spline basis function, γₖ is the k-th coefficient, and cₖ is the k-th control point, and K is the total number of control points of the b-spline function. As a result, the optimization of αᵢ becomes the optimization of γₖ, which can significantly reduce the dimension of unknow variables, leading to a stable and unique solution.

Other forms of basis function also can be used. For example, the change of the tube current can be modelled using a piece-wise constant curve, which is equivalent to the case where the basis function B is replaced with a zero-order piece-wise constant b-spline function. The calculation of view-group reconstruction also can be further simplified, as it can be performed for a group of views that share the same parameters.

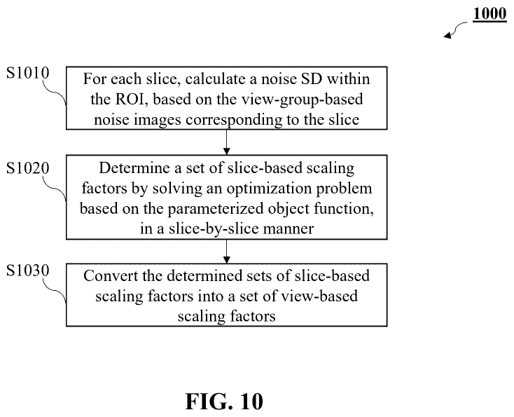

FIG. 10 shows a flow chart of an exemplary procedure for generating a tube current curve in accordance with embodiments of the disclosure. In step S1010, for each slice, a noise SD is calculated within the ROI, based on the view-group-based noise images corresponding to the slice. In step S1020, sets of slice-based scaling factors are determined by solving an optimization problem, in a slice-by-slice manner. The optimization problem can be built based on a basic function that has a limited numbers of unknown variables to enhance the stability of the optimization process. In step S1030, the determined sets of slice-based scaling factors are converted to a set of view-based scaling factors for implementing the AEC modulation.

Compared to simple model or look-up table methods with 2D radiographic images, the embodiments described in this disclosure can provide patient-specific task-dependent AEC prediction, using pre-scan 3D patient information and view-group-based noise image reconstruction. Furthermore, the optimization process uses objective functions parameterized with basis functions, providing a stable and unique solution.

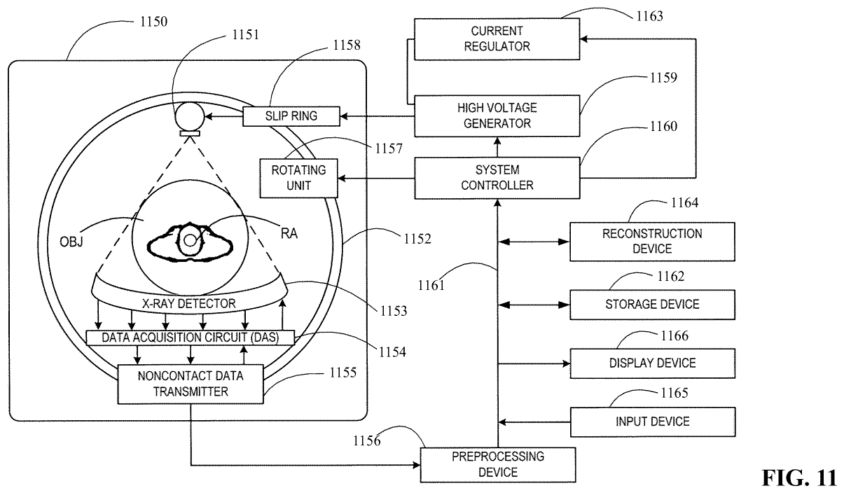

FIG. 11 is a schematic block diagram of a CT apparatus or scanner, according to one embodiment of the present disclosure. As shown in FIG. 11, a radiography gantry 1150 is illustrated from a side view and further includes an X-ray tube 1151, an annular frame 1152, and a multi-row or two-dimensional-array-type X-ray detector 1153. The X-ray tube 1151 and X-ray detector 1153 are diametrically mounted across an object OBJ on the annular frame 1152, which is rotatably supported around a rotation axis RA. A rotating unit 1157 rotates the annular frame 1152 at a high speed, such as 0.4 sec/rotation, while the object OBJ is being moved along the axis RA into or out of the illustrated page.

An embodiment of an X-ray CT apparatus according to the present disclosure will be described below with reference to the views of the accompanying drawing. Note that X-ray CT apparatuses include various types of apparatuses, e.g., a rotate/rotate-type apparatus in which an X-ray tube and X-ray detector rotate together around an object to be examined, and a stationary/rotate-type apparatus in which many detection elements are arrayed in the form of a ring or plane, and only an X-ray tube rotates around an object to be examined. The present disclosure can be applied to either type. In this case, the rotate/rotate-type, which is currently the mainstream, will be exemplified.

The multi-slice X-ray CT apparatus further includes a high voltage generator 1159 that generates a tube voltage applied to the X-ray tube 1151 through a slip ring 1158 so that the X-ray tube 1151 generates X-rays. The X-rays are emitted towards the object OBJ, whose cross-sectional area is represented by a circle. For example, the X-ray tube 1151 having an average X-ray energy during a first scan that is less than an average X-ray energy during a second scan. Thus, two or more scans can be obtained corresponding to different X-ray energies. The X-ray detector 1153 is located at the opposite side from the X-ray tube 1151 across the object OBJ for detecting the emitted X-rays that have transmitted through the object OBJ. The X-ray detector 1153 further includes individual detector elements or units.

The CT apparatus further includes other devices for processing the detected signals from the X-ray detector 1153. A data acquisition circuit or a Data Acquisition System (DAS) 1154 converts a signal output from the X-ray detector 1153 for each channel into a voltage signal, amplifies the signal, and further converts the signal into a digital signal. The X-ray detector 1153 and the DAS 1154 are configured to handle a predetermined total number of projections per rotation (TPPR).

The above-described data is sent to a preprocessing device 1156, which is housed in a console outside the radiography gantry 1150 through a non-contact data transmitter 1155. The preprocessing device 1156 performs certain corrections, such as sensitivity correction, on the raw data. A memory 1162 stores the resultant data, which is also called projection data at a stage immediately before reconstruction processing. The memory 1162 is connected to a system controller 1160 through a data/control bus 1161, together with a reconstruction device 1164, input device 1165, and display 1166. The system controller 1160 controls a current regulator 1163 that limits the current to a level sufficient for driving the CT system.

The detectors are rotated and/or fixed with respect to the patient among various generations of the CT scanner systems. In one implementation, the above-described CT system can be an example of a combined third-generation geometry and fourth-generation geometry system. In the third-generation system, the X-ray tube 1151 and the X-ray detector 1153 are diametrically mounted on the annular frame 1152 and are rotated around the object OBJ as the annular frame 1152 is rotated about the rotation axis RA. In the fourth-generation geometry system, the detectors are fixedly placed around the patient and an X-ray tube rotates around the patient. In an alternative embodiment, the radiography gantry 1150 has multiple detectors arranged on the annular frame 1152, which is supported by a C-arm and a stand.

The memory 1162 can store the measurement value representative of the irradiance of the X-rays at the X-ray detector unit 1153. Further, the memory 1162 can store a dedicated program for executing the CT image reconstruction, material decomposition, and motion estimation and motion compensation methods including the methods described herein.

The reconstruction device 1164 can execute the above-referenced methods, described herein. Further, reconstruction device 1164 can execute pre-reconstruction processing image processing such as volume rendering processing and image difference processing as needed.

The pre-reconstruction processing of the projection data performed by the preprocessing device 1156 can include correcting for detector calibrations, detector nonlinearities, and polar effects, for example.

Post-reconstruction processing performed by the reconstruction device 1164 can include filtering and smoothing the image, volume rendering processing, and image difference processing, as needed. The image reconstruction process can be performed using filtered back projection, iterative image reconstruction methods, or stochastic image reconstruction methods. The reconstruction device 1164 can use the memory to store, e.g., projection data, reconstructed images, calibration data and parameters, and computer programs.

The reconstruction device 1164 can include a CPU (processing circuitry) that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VDHL, Verilog, or any other hardware description language and the code may be stored in an electronic memory directly within the FPGA or CPLD, or as a separate electronic memory. Further, the memory 1162 can be non-volatile, such as ROM, EPROM, EEPROM or FLASH memory. The memory 1162 can also be volatile, such as static or dynamic RAM, and a processor, such as a microcontroller or microprocessor, can be provided to manage the electronic memory as well as the interaction between the FPGA or CPLD and the memory.

Alternatively, the CPU in the reconstruction device 1164 can execute a computer program including a set of computer-readable instructions that perform the functions described herein, the program being stored in any of the above-described non-transitory electronic memories and/or a hard disc drive, CD, DVD, FLASH drive or any other known storage media. Further, the computer-readable instructions may be provided as a utility application, background daemon, or component of an operating system, or combination thereof, executing in conjunction with a processor, such as a Xeon processor from Intel of America or an Opteron processor from AMD of America and an operating system, such as Microsoft 10, UNIX, Solaris, LINUX, Apple, MAC-OS and other operating systems known to those skilled in the art. Further, CPU can be implemented as multiple processors cooperatively working in parallel to perform the instructions.

In one implementation, the reconstructed images can be displayed on a display 1166. The display 1166 can be an LCD display, CRT display, plasma display, OLED, LED or any other display known in the art.

The memory 1162 can be a hard disk drive, CD-ROM drive, DVD drive, FLASH drive, RAM, ROM or any other electronic storage known in the art.

Numerous modifications and variations of the embodiments presented herein are possible in light of the above teachings. It is therefore to be understood that within the scope of the claims, the application may be practiced otherwise than as specifically described herein. The inventions are not limited to the examples that have just been described; it is in particular possible to combine features of the illustrated examples with one another in variants that have not been illustrated.

Embodiments of the present disclosure may also be as set forth in the following parentheticals.

(1) An apparatus for performing X-ray exposure control in a computed tomography (CT) imaging system including an X-ray source, the apparatus comprising: processing circuitry configured to acquire scout scan data from a scout scan performed on an imaging object, in a slice-by-slice manner, generate a plurality of view-group-based noise images for slices of the CT imaging system, based on the acquired scout scan data, identify a region-of-interest (ROI) with respect to which the X-ray exposure control is to be performed, determine a target noise standard deviation (SD) to be achieved by performing the X-ray exposure control, generate a tube current modulation curve with respect to the identified ROI, based on the generated plurality of view-group-based noise images and the determined target noise SD, and perform an imaging scan on the imaging object, with a tube current applied to the X-ray source being modulated based on the generated tube current modulation curve.

(2) The apparatus of (1), wherein the processing circuitry is further configured to generate the plurality of view-group-based noise images by: for each view of the CT imaging system, generating a corresponding noise projection based on the acquired scout scan data, the corresponding noise projection representing a variance in X-ray photon counts measured in the view during the scout scan, and for each slice of the CT imaging system, dividing views that contribute to the slice into a predetermined number of view groups, and for each view group of the predetermined number of view groups, performing reconstruction based on the noise projections generated for the views in the view group, to obtain a noise image corresponding to the view group.

(3) The apparatus of (2), wherein the processing circuitry is further configured to, for each view of the CT imaging system, use a pre-defined noise model to calculate the corresponding noise projection, the pre-defined noise model having a first term and a second term, the first term represents a photon variance in the X-ray photon counts measured in the view during the scout scan, the photon variance following a Poisson distribution, and the second term represents an electronic variance in the X-ray photon counts measured in the view during the scout scan, the electronic variance following a Gaussian distribution.

(4) The apparatus of (1), wherein the processing circuitry is further configured to identify the ROI by: reconstructing an image of the imaging object, based on the acquired scout scan data, segmenting an anatomical structure of the imaging object in the reconstructed image, and determining, based on an input from an operator of the CT imaging system, or based on a predetermined threshold, a region associated with the segmented anatomical structure, as the identified ROI.

(5) The apparatus of (1), wherein the processing circuitry is further configured to generate the tube current modulation curve by: for each slice of the CT imaging system, calculating a noise SD within the ROI, based on the predetermined number of view-group-based noise images generated for the slice, and determining a set of slice-based scaling factors, by solving an optimization function to match the calculated noise SD with the determined target noise SD, and converting the sets of slice-based scaling factors determined for the slices of the CT imaging system to a set of view-based scaling factors, as the generated tube current modulation curve, each scaling factor in the converted set of view-based scaling factors corresponding to a specific view of the CT imaging system.

(6) The apparatus of (5), wherein the processing circuitry is further configured to derive an objective function parametrized with a pre-defined basis function, as the optimization function, the pre-defined basis function having a predetermined number of variables.

(7) The apparatus of (6), wherein the processing circuitry is further configured to derive an objective function parametrized with a b-spline function, as the optimization function.

(8) A method for performing X-ray exposure control in a computed tomography (CT) imaging system including an X-ray source, the method comprising: acquiring scout scan data from a scout scan performed on an imaging object; in a slice-by-slice manner, generating a plurality of view-group-based noise images for slices of the CT imaging system, based on the acquired scout scan data; identifying a region-of-interest (ROI) with respect to which the X-ray exposure control is to be performed; determining a target noise standard deviation (SD) to be achieved by performing the X-ray exposure control; generating a tube current modulation curve with respect to the identified ROI, based on the generated plurality of view-group-based noise images and the determined target noise SD; and performing an imaging scan on the imaging object, with a tube current applied to the X-ray source being modulated based on the generated tube current modulation curve.

(9) The method of (8), wherein the step of generating the plurality of view-group-based noise images further comprises: for each view of the CT imaging system, generating a corresponding noise projection based on the acquired scout scan data, the corresponding noise projection representing a variance in X-ray photon counts measured in the view during the scout scan, and for each slice of the CT imaging system, dividing views that contribute to the slice into a predetermined number of view groups, and for each view group of the predetermined number of view groups, performing reconstruction based on the noise projections generated for the views in the view group, to obtain a noise image corresponding to the view group.

(10) The method of (9), wherein the step of generating the corresponding noise projection further comprises: using a pre-defined noise model to calculate the corresponding noise projection, the pre-defined noise model having a first term and a second term, the first term represents a photon variance in the X-ray photon counts measured in the view during the scout scan, the photon variance following a Poisson distribution, and the second term represents an electronic variance in the X-ray photon counts measured in the view during the scout scan, the electronic variance following a Gaussian distribution.

(11) The method of (8), wherein the step of identifying the ROI further comprises: reconstructing an image of the imaging object, based on the acquired scout scan data, segmenting an anatomical structure of the imaging object in the reconstructed image, and determining, based on an input from an operator of the CT imaging system, or based on a predetermined threshold, a region associated with the segmented anatomical structure, as the identified ROI.

(12) The method of (8), wherein the step of generating the tube current modulation curve by: for each slice of the CT imaging system, calculating a noise SD within the ROI, based on the predetermined number of view-group-based noise images generated for the slice, and determining a set of slice-based scaling factors, by solving an optimization function to match the calculated noise SD with the determined target noise SD, and converting the sets of slice-based scaling factors determined for the slices of the CT imaging system to a set of view-based scaling factors, as the generated tube current modulation curve, each scaling factor in the converted set of view-based scaling factors corresponding to a specific view of the CT imaging system.

(13) The method of (12), further comprising deriving an objective function parametrized with a pre-defined basis function, as the optimization function, the pre-defined basis function having a predetermined number of variables.

(14) The method of (13), further comprising deriving an objective function parametrized with a b-spline function, as the optimization function.

(15) A non-transitory computer readable medium having instructions stored therein that, when executed by one or more processors, cause the one or more processors to perform a method for performing X-ray exposure control in a computed tomography (CT) imaging system including an X-ray source, the method comprising: acquiring scout scan data from a scout scan performed on an imaging object; in a slice-by-slice manner, generating a plurality of view-group-based noise images for slices of the CT imaging system, based on the acquired scout scan data; identifying a region-of-interest (ROI) with respect to which the X-ray exposure control is to be performed; determining a target noise standard deviation (SD) to be achieved by performing the X-ray exposure control; generating a tube current modulation curve with respect to the identified ROI, based on the generated plurality of view-group-based noise images and the determined target noise SD; and performing an imaging scan on the imaging object, with a tube current applied to the X-ray source being modulated based on the generated tube current modulation curve.

(16) The non-transitory computer readable medium of (15), wherein the step of generating the plurality of view-group-based noise images further comprises: for each view of the CT imaging system, generating a corresponding noise projection based on the acquired scout scan data, the corresponding noise projection representing a variance in X-ray photon counts measured in the view during the scout scan, and for each slice of the CT imaging system, dividing views that contribute to the slice into a predetermined number of view groups, and for each view group of the predetermined number of view groups, performing reconstruction based on the noise projections generated for the views in the view group, to obtain a noise image corresponding to the view group.

(17) The non-transitory computer readable medium of (16), wherein the step of generating the corresponding noise projection further comprises: using a pre-defined noise model to calculate the corresponding noise projection, the pre-defined noise model having a first term and a second term, the first term represents a photon variance in the X-ray photon counts measured in the view during the scout scan, the photon variance following a Poisson distribution, and the second term represents an electronic variance in the X-ray photon counts measured in the view during the scout scan, the electronic variance following a Gaussian distribution.

(18) The non-transitory computer readable medium of (15), wherein the step of identifying the ROI further comprises: reconstructing an image of the imaging object, based on the acquired scout scan data, segmenting an anatomical structure of the imaging object in the reconstructed image, and determining, based on an input from an operator of the CT imaging system, or based on a predetermined threshold, a region associated with the segmented anatomical structure, as the identified ROI.

(19) The non-transitory computer readable medium of (15), wherein the step of generating the tube current modulation curve by: for each slice of the CT imaging system, calculating a noise SD within the ROI, based on the predetermined number of view-group-based noise images generated for the slice, and determining a set of slice-based scaling factors, by solving an optimization function to match the calculated noise SD with the determined target noise SD, and converting the sets of slice-based scaling factors determined for the slices of the CT imaging system to a set of view-based scaling factors, as the generated tube current modulation curve, each scaling factor in the converted set of view-based scaling factors corresponding to a specific view of the CT imaging system.

(20) The non-transitory computer readable medium of (19), wherein the method further comprises deriving an objective function parametrized with a pre-defined basis function, as the optimization function, the pre-defined basis function having a predetermined number of variables.

Numerous modifications and variations of the embodiments presented herein are possible in light of the above teachings. It is therefore to be understood that within the scope of the claims, the disclosure may be practiced otherwise than as specifically described herein.

What is claimed is:

1. An apparatus for performing X-ray exposure control in a computed tomography (CT) imaging system including an X-ray source, the apparatus comprising:
processing circuitry configured to
acquire scout scan data from a scout scan performed on an imaging object,
in a slice-by-slice manner, generate a plurality of view-group-based noise images for slices of the CT imaging system, based on the acquired scout scan data,
identify a region-of-interest (ROI) with respect to which the X-ray exposure control is to be performed,
determine a target noise standard deviation (SD) to be achieved by performing the X-ray exposure control,
generate a tube current modulation curve with respect to the identified ROI, based on the generated plurality of view-group-based noise images and the determined target noise SD, and
perform an imaging scan on the imaging object, with a tube current applied to the X-ray source being modulated based on the generated tube current modulation curve.

2. The apparatus of claim 1, wherein the processing circuitry is further configured to generate the plurality of view-group-based noise images by:
for each view of the CT imaging system, generating a corresponding noise projection based on the acquired scout scan data, the corresponding noise projection representing a variance in X-ray photon counts measured in the view during the scout scan, and
for each slice of the CT imaging system,
dividing views that contribute to the slice into a predetermined number of view groups, and
for each view group of the predetermined number of view groups, performing reconstruction based on the noise projections generated for the views in the view group, to obtain a noise image corresponding to the view group.

3. The apparatus of claim 2, wherein the processing circuitry is further configured to, for each view of the CT imaging system, use a pre-defined noise model to calculate the corresponding noise projection, the pre-defined noise model having a first term and a second term,
the first term represents a photon variance in the X-ray photon counts measured in the view during the scout scan, the photon variance following a Poisson distribution, and
the second term represents an electronic variance in the X-ray photon counts measured in the view during the scout scan, the electronic variance following a Gaussian distribution.

4. The apparatus of claim 1, wherein the processing circuitry is further configured to identify the ROI by:
reconstructing an image of the imaging object, based on the acquired scout scan data,
segmenting an anatomical structure of the imaging object in the reconstructed image, and
determining, based on an input from an operator of the CT imaging system, or based on a predetermined threshold, a region associated with the segmented anatomical structure, as the identified ROI.

5. The apparatus of claim 1, wherein the processing circuitry is further configured to generate the tube current modulation curve by:
for each slice of the CT imaging system,
calculating a noise SD within the ROI, based on the predetermined number of view-group-based noise images generated for the slice, and
determining a set of slice-based scaling factors, by solving an optimization function to match the calculated noise SD with the determined target noise SD, and
converting the sets of slice-based scaling factors determined for the slices of the CT imaging system to a set of view-based scaling factors, as the generated tube current modulation curve, each scaling factor in the converted set of view-based scaling factors corresponding to a specific view of the CT imaging system.

6. The apparatus of claim 5, wherein the processing circuitry is further configured to derive an objective function parametrized with a pre-defined basis function, as the optimization function, the pre-defined basis function having a predetermined number of variables.

7. The apparatus of claim 6, wherein the processing circuitry is further configured to derive an objective function parametrized with a b-spline function, as the optimization function.

8. A method for performing X-ray exposure control in a computed tomography (CT) imaging system including an X-ray source, the method comprising:
acquiring scout scan data from a scout scan performed on an imaging object;
in a slice-by-slice manner, generating a plurality of view-group-based noise images for slices of the CT imaging system, based on the acquired scout scan data;
identifying a region-of-interest (ROI) with respect to which the X-ray exposure control is to be performed;
determining a target noise standard deviation (SD) to be achieved by performing the X-ray exposure control;
generating a tube current modulation curve with respect to the identified ROI, based on the generated plurality of view-group-based noise images and the determined target noise SD; and
performing an imaging scan on the imaging object, with a tube current applied to the X-ray source being modulated based on the generated tube current modulation curve.

9. The method of claim 8, wherein the step of generating the plurality of view-group-based noise images further comprises:
for each view of the CT imaging system, generating a corresponding noise projection based on the acquired scout scan data, the corresponding noise projection representing a variance in X-ray photon counts measured in the view during the scout scan, and
for each slice of the CT imaging system,
dividing views that contribute to the slice into a predetermined number of view groups, and
for each view group of the predetermined number of view groups, performing reconstruction based on the noise projections generated for the views in the view group, to obtain a noise image corresponding to the view group.

10. The method of claim 9, wherein the step of generating the corresponding noise projection further comprises: using a pre-defined noise model to calculate the corresponding noise projection, the pre-defined noise model having a first term and a second term,
the first term represents a photon variance in the X-ray photon counts measured in the view during the scout scan, the photon variance following a Poisson distribution, and
the second term represents an electronic variance in the X-ray photon counts measured in the view during the scout scan, the electronic variance following a Gaussian distribution.

11. The method of claim 8, wherein the step of identifying the ROI further comprises:
reconstructing an image of the imaging object, based on the acquired scout scan data,
segmenting an anatomical structure of the imaging object in the reconstructed image, and
determining, based on an input from an operator of the CT imaging system, or based on a predetermined threshold, a region associated with the segmented anatomical structure, as the identified ROI.

12. The method of claim 8, wherein the step of generating the tube current modulation curve by:
for each slice of the CT imaging system,
calculating a noise SD within the ROI, based on the predetermined number of view-group-based noise images generated for the slice, and
determining a set of slice-based scaling factors, by solving an optimization function to match the calculated noise SD with the determined target noise SD, and
converting the sets of slice-based scaling factors determined for the slices of the CT imaging system to a set of view-based scaling factors, as the generated tube current modulation curve, each scaling factor in the converted set of view-based scaling factors corresponding to a specific view of the CT imaging system.

13. The method of claim 12, further comprising deriving an objective function parametrized with a pre-defined basis function, as the optimization function, the pre-defined basis function having a predetermined number of variables.

14. The method of claim 13, further comprising deriving an objective function parametrized with a b-spline function, as the optimization function.

15. A non-transitory computer readable medium having instructions stored therein that, when executed by one or more processors, cause the one or more processors to perform a method for performing X-ray exposure control in a computed tomography (CT) imaging system including an X-ray source, the method comprising:
acquiring scout scan data from a scout scan performed on an imaging object;
in a slice-by-slice manner, generating a plurality of view-group-based noise images for slices of the CT imaging system, based on the acquired scout scan data;
identifying a region-of-interest (ROI) with respect to which the X-ray exposure control is to be performed;
determining a target noise standard deviation (SD) to be achieved by performing the X-ray exposure control;
generating a tube current modulation curve with respect to the identified ROI, based on the generated plurality of view-group-based noise images and the determined target noise SD; and
performing an imaging scan on the imaging object, with a tube current applied to the X-ray source being modulated based on the generated tube current modulation curve.

16. The non-transitory computer readable medium of claim 15, wherein the step of generating the plurality of view-group-based noise images further comprises:
for each view of the CT imaging system, generating a corresponding noise projection based on the acquired scout scan data, the corresponding noise projection representing a variance in X-ray photon counts measured in the view during the scout scan, and
for each slice of the CT imaging system,
dividing views that contribute to the slice into a predetermined number of view groups, and
for each view group of the predetermined number of view groups, performing reconstruction based on the noise projections generated for the views in the view group, to obtain a noise image corresponding to the view group.

17. The non-transitory computer readable medium of claim 16, wherein the step of generating the corresponding noise projection further comprises: using a pre-defined noise model to calculate the corresponding noise projection, the pre-defined noise model having a first term and a second term,
the first term represents a photon variance in the X-ray photon counts measured in the view during the scout scan, the photon variance following a Poisson distribution, and
the second term represents an electronic variance in the X-ray photon counts measured in the view during the scout scan, the electronic variance following a Gaussian distribution.

18. The non-transitory computer readable medium of claim 15, wherein the step of identifying the ROI further comprises:
reconstructing an image of the imaging object, based on the acquired scout scan data,
segmenting an anatomical structure of the imaging object in the reconstructed image, and
determining, based on an input from an operator of the CT imaging system, or based on a predetermined threshold, a region associated with the segmented anatomical structure, as the identified ROI.

19. The non-transitory computer readable medium of claim 15, wherein the step of generating the tube current modulation curve by:
for each slice of the CT imaging system,
calculating a noise SD within the ROI, based on the predetermined number of view-group-based noise images generated for the slice, and
determining a set of slice-based scaling factors, by solving an optimization function to match the calculated noise SD with the determined target noise SD, and
converting the sets of slice-based scaling factors determined for the slices of the CT imaging system to a set of view-based scaling factors, as the generated tube current modulation curve, each scaling factor in the converted set of view-based scaling factors corresponding to a specific view of the CT imaging system.

20. The non-transitory computer readable medium of claim 19, wherein the method further comprises deriving an objective function parametrized with a pre-defined basis function, as the optimization function, the pre-defined basis function having a predetermined number of variables.
